(19) United States
(12) Patent Application Publication
(10) Pub. No.: US 2020/0037889 A1
(43) Pub. Date: Feb. 6, 2020

(54) Blood Pressure Measurement Device and Blood Pressure Measurement Method

(71) Applicant: AViTA Corporation, New Taipei City (TW)

(72) Inventors: Jui-Yang Huang, New Taipei City (TW); I-Chih Huang, New Taipei City (TW)

(21) Appl. No.: 16/528,531

(22) Filed: Jul. 31, 2019

(30) Foreign Application Priority Data
Jul. 31, 2018 (TW) 107126407

(51) Int. Cl. A61B 5/022; A61B 5/021

(52) U.S. Cl. CPC A61B 5/022; A61B 5/02108; A61B 5/02007; A61B 2560/0223; A61B 5/02141

(57) ABSTRACT

A blood pressure measurement method and a blood pressure measurement device using the same are provided. The method includes identifying an input value; if the input value is larger than 2, performing, by a blood pressure detector, N times of blood pressure measurement operation sequentially according to the input value, so as to obtain N blood pressure detection values, wherein a value of N is equal to the input value; sorting, according to the size of the N blood pressure detection values, the N blood pressure detection values to obtain N sorted blood detection values, and selecting one or more target sorted blood detection values which is ordered in the middle of the N sorted blood pressure detection values, so as to obtain a blood pressure measurement result value; and outputting, by an output unit, the blood pressure measurement result value.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

### Field of Invention

The present invention relates to a measurement device, and more particularly to a blood pressure measurement device and a blood pressure measurement method.

### Description of Related Arts

In general, a conventional blood pressure machine, which is a device for measuring blood pressure, can perform multiple blood pressure detection operations on a measurer, to obtain a plurality of blood pressure detection values, and take an arithmetic mean of all blood pressure detection values as a final blood pressure measurement result for output and display. However, in some specific cases, the measured blood pressure detection value may be too high or too low. For example, the specific case can be that the user has insufficient rest when measuring the blood pressure; or, during the measurement process the user's posture is incorrect, for example, an air bladder of the blood pressure machine is not at the same height as the user's heart or the air bladder is shaking during the measurement process; or, the measurement process is affected by an external environmental factor such as noise.

The excessively high or low blood pressure detection value measured in these specific cases possibly distorts the final blood pressure measurement result. For example, it is assumed that the conventional blood pressure machine automatically performs three blood pressure measurement operations to obtain three blood pressure detection values such as 85, 180 and 95, and calculates the arithmetic mean of the three blood pressure detection values to obtain a final blood pressure measurement result, which is 120 (85+180+95)/3=120). If one of the blood pressure detection values is too high (such as 180), the final blood pressure measurement results (such as 120) obtained by the above-mentioned conventional method possibly deviates too much from the blood pressure detection values measured in other normal case, such as 85 or 95. As a result, the excessively high or low blood pressure detection value may lead to misjudgment of the blood pressure of the user/subject.

Therefore, how to solve the defect of the conventional blood pressure machine to obtain a more accurate blood pressure measurement result, is an important issue in the industry.

### SUMMARY OF THE PRESENT INVENTION

The present invention provides a blood pressure measurement device and a blood pressure measurement method which is able to obtain a more accurate blood pressure measurement result by performing multiple blood pressure detection operations.

According to an embodiment, the present invention provides a blood pressure measurement device comprising a control unit, a detector and an output unit. The detector is coupled to the control unit and configured to measure a blood pressure of a user. The output unit is coupled to the control unit and configured to output information. The control unit is configured to identify a first parameter, and the first parameter indicates the number of times for which a blood pressure detection operation should be executed. When the first parameter is higher than 2, the control unit instructs the detector to sequentially execute the blood pressure detection operation for N times according to the first parameter, to obtain the N blood pressure detection values, and a value of N is equal to the first parameter, and the control unit sorts the N blood pressure detection values by value to obtain N sorted blood pressure detection values, and selects at least one target sorted blood pressure detection value which is arranged in the middle of the N sorted blood pressure detection values, so as to obtain a blood pressure measurement result value, and the control unit instructs the output unit to output the blood pressure measurement result value.

According to an embodiment, the present invention provides a blood pressure measurement method comprising following step: identifying a first parameter, wherein the first parameter indicates the number of times for which a blood pressure detection operation should be executed; when the first parameter is higher than 2, using a detector to sequentially execute the blood pressure detection operation for N times according to the first parameter to obtain N blood pressure detection values, wherein a value of N is equal to the first parameter; using a control unit to sort the N blood pressure detection values by value to obtain N sorted blood pressure detection values, and selecting at least one target sorted blood pressure detection value which is arranged in the middle of the N sorted blood pressure detection values, so as to obtain a blood pressure measurement result value; and using an output unit to output the blood pressure measurement result value.

According to aforementioned content, the blood pressure measurement device and blood pressure measurement method of the embodiments of the present invention can perform multiple blood pressure detection operations, to obtain the multiple blood pressure detection values, and then sort the multiple blood pressure detection values to obtain the multiple sorted blood pressure detection values, and then select at least one target sorted blood pressure detection value which is arranged in the middle of the multiple sorted blood pressure detection values, so as to obtain the blood pressure measurement result value. As a result, the problem of a deviation in blood pressure measurement result caused by conventional method can be prevented.

### BRIEF DESCRIPTION OF THE DRAWINGS

The structure, operating principle and effects of the present invention will be described in detail by way of various embodiments which are illustrated in the accompanying drawings.

FIG. 1A is a block diagram of a blood pressure measurement device of an embodiment of the present invention.

FIG. 1B is a block diagram of a detector of an embodiment of the present invention.

FIG. 2A is a flowchart of a blood pressure measurement method of an embodiment of the present invention.

FIG. 2B is a flowchart of a step S22 of FIG. 2A according to an embodiment of the present invention.

FIG. 3 is a flowchart of a blood pressure measurement method of another embodiment of the present invention.

### DETAILED DESCRIPTION OF THE PREFERRED EMBODIMENT

The following embodiments of the present invention are herein described in detail with reference to the accompanying drawings. These drawings show specific examples of the embodiments of the present invention. These embodiments are provided so that this disclosure will be thorough and complete, and will fully convey the scope of the invention to those skilled in the art. It is to be acknowledged that these embodiments are exemplary implementations and are not to be construed as limiting the scope of the present invention in any way. Further modifications to the disclosed embodiments, as well as other embodiments, are also included within the scope of the appended claims. These embodiments are provided so that this disclosure is thorough and complete, and fully conveys the inventive concept to those skilled in the art. Regarding the drawings, the relative proportions and ratios of elements in the drawings may be exaggerated or diminished in size for the sake of clarity and convenience. Such arbitrary proportions are only illustrative and not limiting in any way. The same reference numbers are used in the drawings and description to refer to the same or like parts.

It is to be acknowledged that although the terms ‘first’, ‘second’, ‘third’, and so on, may be used herein to describe various elements, these elements should not be limited by these terms. These terms are used only for the purpose of distinguishing one component from another component. Thus, a first element discussed herein could be termed a second element without altering the description of the present disclosure. As used herein, the term “or” includes any and all combinations of one or more of the associated listed items.

It will be acknowledged that when an element or layer is referred to as being “on,” “connected to” or “coupled to” another element or layer, it can be directly on connected or coupled to the other element or layer, or intervening elements or layers may be present. In contrast, when an element is referred to as being “directly on,” “directly connected to” or “directly coupled to” another element or layer, there are no intervening elements or layers present.

In addition, unless explicitly described to the contrary, the word “comprise” and variations such as “comprises” or “comprising”, will be acknowledged to imply the inclusion of stated elements but not the exclusion of any other elements.

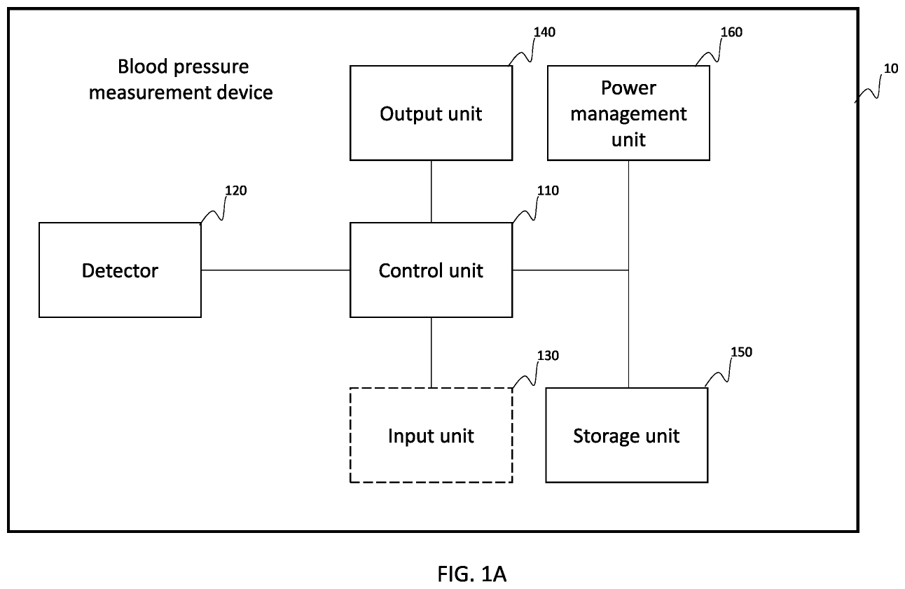

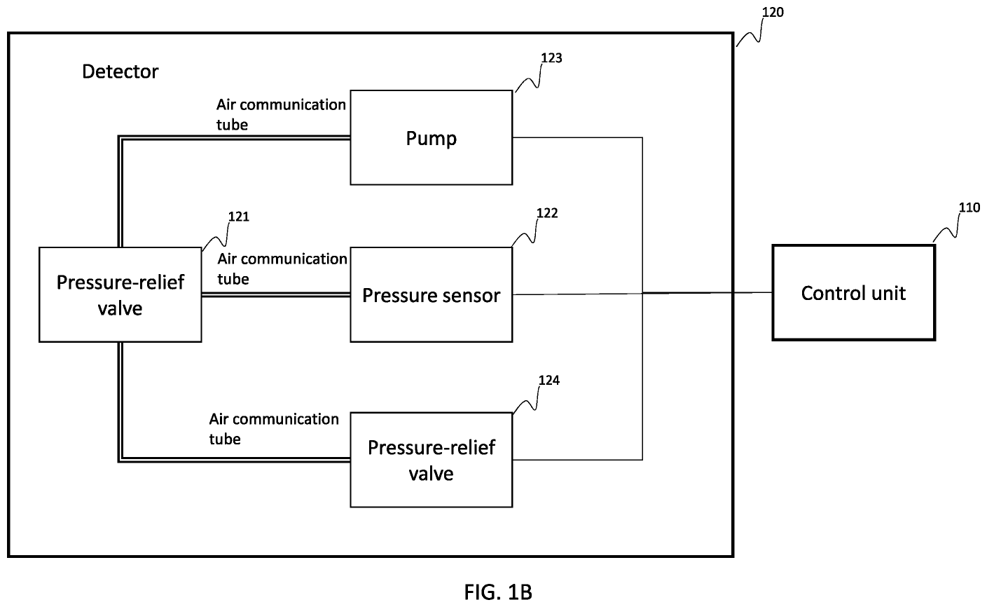

FIG. 1A is a block diagram of a blood pressure measurement device of an embodiment of the present invention. As shown in FIG. 1A, in this embodiment, a blood pressure measurement device 10 can include a control unit 110, a detector 120, an output unit 140, a storage unit 150 and a power management unit 160. In an embodiment, the blood pressure measurement device 10 can include an input unit 130.

The control unit 110 can be a hardware unit with computing capability, such as a chipset and a processor, and can manage overall operations of the blood pressure measurement device 10 and transmit different signals to the detector 120, to execute a blood pressure detection operation. After each blood pressure detection operation is completed, the detector 120 transmits a corresponding blood pressure detection value, to the control unit 110. The control unit 110 can temporarily store the received blood pressure detection value into the storage unit 150. In this embodiment, the control unit 110 can be, for example, a single-core or multi-core central control unit, a micro-processor, or other programmable control unit, a digital signal processor, a programmable controller, an application-specific integrated circuit (ASIC), a programmable logic device (PLD), or other similar devices. In an embodiment, the control unit 110 can comprise a blood pressure detection value management circuit unit configured to calculate a blood pressure measurement result value according to the blood pressure detection value received from the detector 120.

The input unit 130 is coupled to the control unit 110. In an embodiment, the input unit 130 can be a mouse, a keyboard, a scanner, a digital camera, a digital tablet, a microphone, a touch pad or a touch panel, and the input unit 130 provides a user to input data or to control an operating system which the user wants to operate, through the input unit 130. The output unit 140 can be a screen which receives display data to display an image, or a speaker which receives audio data to make sound, or the like; however, the present invention is not limited to above-mentioned examples. In an embodiment, the input unit 130 can be integrated to the output unit 140, for example, a touch screen can display (output) data (such as an image or a text), and also can receive the user's touch operation to input data (such as a first parameter).

The storage unit 150 is coupled to the control unit 110 and configured to store data. The storage unit 150 can be any type of hard disk drive (HDD) or non-volatile memory storage device. The data can be, for example, a standard blood pressure measurement result value, software or firmware used to manage blood pressure measurement device 10, or historical statistics data that must be stored for a long time. For example, the historical statistics data can be each user's historical data of blood pressure measurement results. The standard blood pressure measurement result value record corresponds to the different type of the blood pressure measurement result value, and the different types can include different ages, genders, or different blood pressure types such as systolic and diastolic blood pressures. In other embodiments, the storage unit 150 can also be integrated into the control unit 110.

The power management unit 160 can be used to manage power of the blood pressure measurement device 10. Furthermore, the power management unit 160 can be connected to an external power supply or a built-in battery, so as to provide the power required for the overall operations of the blood pressure measurement device 10.

The details of the detector 120 are explained below with reference to FIG. 1B.

Please refer to FIG. 1B, which is a block diagram of a detector of an embodiment of the present invention. As shown in FIG. 1B, in this embodiment, the detector 120 can include a pressure sensor 122, a pump 123, and a pressure-relief valve 124. An air bladder 121 is connected to the pump 123, the pressure sensor 122, the pressure-relief valve 124 through a plurality of air communication tubes. In an embodiment, the pressure-relief valve 124 or the pump 123 can be directly connected to the air bladder 121. Furthermore, the control unit 110 is coupled to the pump 123, the pressure sensor 122 and the pressure-relief valve 124.

The pump 123 can receive a pressure signal transmitted by the control unit 110, to inflate the air bladder 121. The pressure-relief valve 124 can receive a pressure-relief signal transmitted by the control unit 110, to release a gas pressure of the air bladder 121. The pressure sensor 122 can receive the gas pressure of the air bladder 121 through the air communication tube. The gas pressure can make a pressure sensing element inside the pressure sensor 122 deform to reflect and output a voltage corresponding to the gas pressure. The pressure sensor 122 can calculate a blood pressure value according to a change in the voltage, and output a blood pressure value, which is used as a blood pressure detection value, to the control unit 110. The other details of the detector 120 are well known to those skilled in the art and are not the technologies to be described in the present invention, so detailed descriptions are omitted herein.

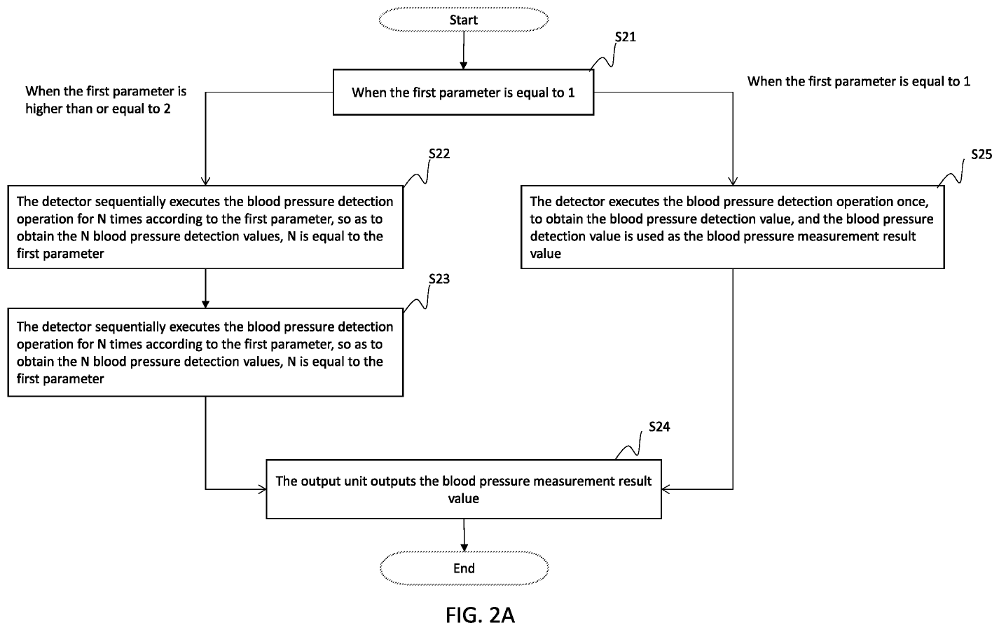

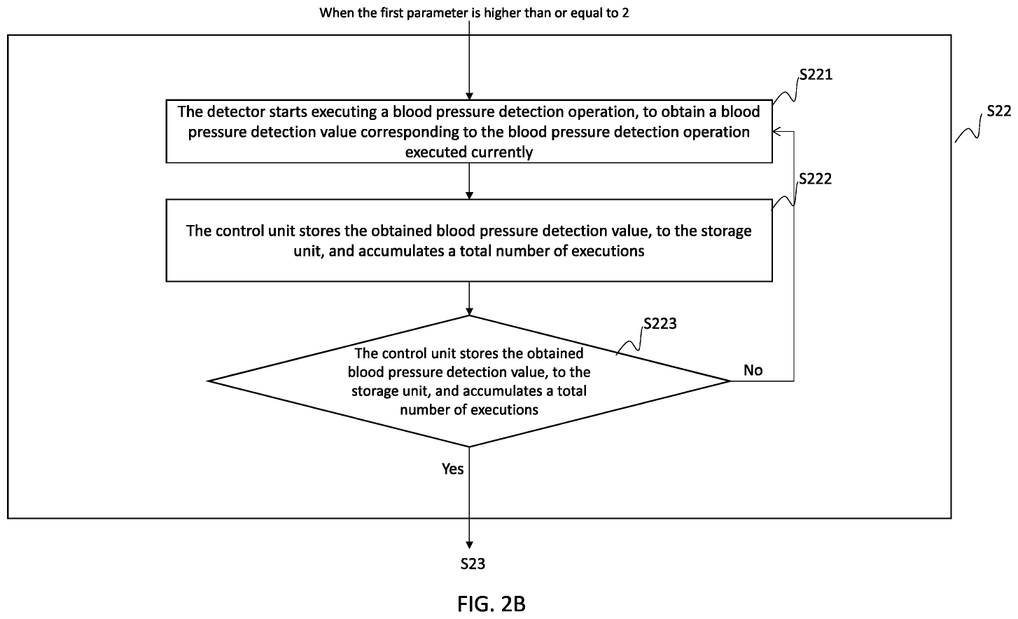

FIG. 2A is a flowchart of a blood pressure measurement method of an embodiment of the present invention.

Please refer to FIG. 2A. In a step S21, the control unit 110 is used to identify the first parameter. For example, the first parameter can be inputted through the input unit 130 such as a switch or a button, so that a user/subject can select a single-measurement blood pressure detection operation mode or a multiple-measurement blood pressure detection operation mode, through the input operation of triggering the switch or button. In response to the operation of triggering the switch or button for selecting the single measurement mode, the identified first parameter is “1”; in response to the operation of triggering the switch or button for selecting the multiple measurement mode, the identified first parameter is indicated as “N”, and N is a number of times for which the blood pressure detection operations should be executed in the multiple-measurement mode, and N is a positive integer higher than 2. The user can use the input unit to set a specific value of N, and in the multiple-measurement mode, the inputted N is limited to a positive integer higher than 2. In another embodiment, the first parameter can also be preset by a manufacturer. The control unit 110 can identify the first parameter inputted by the user, or the first parameter preset by the manufacturer.

In a step S22, when the identified first parameter is higher than 2, for example, the user/subject instructs, by the input operation, the blood pressure measurement device 10 to execute the blood pressure detection operation for multiple times, the detector sequentially executes the blood pressure detection operation for N times according to the first parameter, to obtain the N blood pressure detection values. In other words, after the user applies the input operation on the input unit to set the number of times (that is, the first parameter) for which the blood pressure detection operations should be executed, the control unit 110 can instruct the detector 120 to sequentially execute the blood pressure detection operations for the number of times set through the input unit.

Please refer to FIG. 2B, which is a flowchart of the step S22 of FIG. 2A according to an embodiment of the present invention.

As shown in FIG. 2B, in a step S221, the detector 120 starts executing a blood pressure detection operation, to obtain a blood pressure detection value corresponding to the blood pressure detection operation executed currently. In other words, every time the blood pressure detection operation is completed, the detector 120 outputs the blood pressure detection value corresponding to the blood pressure detection operation completed this time, to the control unit 110.

Next, in a step S222, the control unit stores the obtained blood pressure detection value, to the storage unit, and accumulates a total number of executions. In other words, in response to the completion of this blood pressure detection operation, after receiving the blood pressure detection value, the control unit 110 temporarily stores the blood pressure detection value to the storage unit 150 and adds the number of this execution to the number of blood pressure detection operations executed already, which is a total number of executions. The control unit 110 can use a built-in counter to execute the accumulation operation.

In a step S223, the control unit determines whether the total number of executions is equal to N. In other words, after completing the accumulation of the total number of executions, the control unit 110 checks whether the total number of executions is equal to the first parameter, which is the number of times for which the operations should be executed.

When the total number of executions is equal to N, the step S22 is completed and the flow of the blood pressure measurement method continues to a step S23. When the total number of executions is not equal to N, the flow of the blood pressure measurement method returns to the step S221 and the control unit 110 instructs the detector 120 to start another blood pressure detection operation. In other words, the control unit 110 can instruct the detector 120 to successively execute the blood pressure detection operation by multiple times until the total number of executions is equal to the number of timed for which the detection operation should be executed, which is the first parameter N.

According to the above process, the control unit 110 can be in cooperation with the detector 120 to execute the blood pressure detection operation for multiple times, and temporarily store the obtained blood pressure detection values, and the total number of the executed blood pressure detection operations is equal to the number of times for which the detection operations should be executed.

Please refer back to FIG. 2A. In the step S23, the control unit 110 sorts the N blood pressure detection values by value, to obtain the N sorted blood pressure detection values, and selects at least one target sorted blood pressure detection value which is arranged in the middle of the N sorted blood pressure detection values, so as to obtain the blood pressure measurement result value.

More specifically, in this embodiment, the control unit 110 can first sort the stored N blood pressure detection values by value, for example, from large to small or from small to large, so as to obtain a blood pressure detection value sequence including the N sorted blood pressure detection values. Next, the control unit 110 can determine a median of the N sorted blood pressure detection values by a median method, and take the determined median as the blood pressure measurement result value.

In more detail, when the first parameter is an even value, the control unit 110 can select a plurality of sorted blood pressure detection values arranged in the middle of the N sorted blood pressure detection values, as two target sorted blood pressure detection values, for example, the control unit 10 can select the two sorted blood pressure detection value arranged in the middle of the sorted blood pressure detection values, and use an average of the target sorted blood pressure detection values as the blood pressure measurement result value. Alternatively, when the first parameter is an odd value, the control unit 110 can select a sorted blood pressure detection value arranged in the middle of the N sorted blood pressure detection values, as a target sorted blood pressure detection value, and the control unit 110 can use the target sorted blood pressure detection value as the blood pressure measurement result value.

For example, it is assumed that the first parameter is 4, that is N=4, and the sorted blood pressure detection values are “90”, “95”, “105”, and “110”, the selected target sorted blood pressure detection values are “95” and “105” and the blood pressure measurement result value is “100”, that is, (95+105)/2=100.

For example, it is assumed the first parameter is 3, that is N=3, and the sorted blood pressure detection values are “90”, “95”, and “105”, the selected target sorted blood pressure detection value is “95” and the blood pressure measurement result value is “95”.

In another embodiment, different blood pressure measurement result value calculation manner can be performed according to the first parameter being an odd. More specifically, in another embodiment, the control unit 110 can determine whether the first parameter is an odd value less than 5. When the first parameter is an odd less than 5, the control unit can select a target sorted blood pressure detection value which is arranged in the middle of the N sorted blood pressure detection values, and use the target sorted blood pressure detection value as the blood pressure measurement result value. When the first parameter is an odd value of not less than 5, the control unit can select three target sorted blood pressure detection values which are arranged in the middle of the N sorted blood pressure detection values, and use an average of the three target sorted blood pressure detection values as the blood pressure measurement result value. For example, it is assumed the first parameter is 5, that is N=5, and the sorted blood pressure detection values are “60”, “95”, “100”, “105”, and “120”, the selected three target sorted blood pressure detection values arranged in the middle are “95”, “100”, and “105”, and the blood pressure measurement result value is “100”, that is, (95+100+105)/3=100.

In other words, in another embodiment, when first parameter is higher than or equal to 5, the target sorted blood pressure detection values arranged in the middle portion are selected for further averaging operation, but all blood pressure detection values are not used in the averaging operation. The average of the target sorted blood pressure detection values can be used as the blood pressure measurement result value. As a result, the present invention can combine the advantages of the median and the average calculations for better performance. In other words, the blood pressure detection values near the median of the sorted blood pressure detection values can be selected for averaging operation, it means that the blood pressure detection values arranged in the middle of the sorted blood pressure detection values are selected, and the concept of using the median can prevent influence of an extreme value, and the arithmetic averaging operation only performed on the target blood pressure detection values can obtain the blood pressure measurement result value which can reflect an actual blood pressure of the user/subject.

In a step S24, after the blood pressure measurement result value is obtained, the output unit 140 outputs the blood pressure measurement result value. More specifically, the control unit 110 can instruct the output unit, such as a screen, to display the blood pressure measurement result value. The control unit 110 can record the blood pressure measurement result values obtained in all operations, as historical data of the blood pressure measurement result values of the user. Furthermore, the control unit can compare the blood pressure measurement result value with a standard blood pressure measurement result value, to output a prompt message corresponding a comparison result. For example, when a difference between the obtained blood pressure measurement result value and the standard blood pressure measurement result value is not higher than a predetermined threshold, the control unit 110 indicates the output unit 140 to output a message indicating a normal state; otherwise, when a difference between the obtained blood pressure measurement result value and the standard blood pressure measurement result value is higher than the predetermined threshold, the control unit 110 indicates the output unit 140 to output a message indicating an abnormal state.

On the other hand, when the first parameter identified in the step S21 is 1, for example, the user/subject instructs, by the input operation, the blood pressure measurement device 10 to execute single blood pressure detection operation, the detector 120 executes the blood pressure detection operation once in the step S25, so as to obtain the blood pressure detection value, and the control unit 110 uses the blood pressure detection value as the blood pressure measurement result value.

For example, it is assumed that the three obtained blood pressure detection values are “85” and “180” and “95”, the value of “180” is an abnormal extreme value, and in this case, the blood pressure measurement result value obtained by the conventional method is “120”; in contrast, the blood pressure measurement result value obtained by the blood pressure measurement method of an embodiment of the present invention is “95”. In a condition that the standard blood pressure measurement result value is “90”, the blood pressure measurement result value obtained by the conventional method is more likely determined as an abnormal value, compared with the blood pressure measurement result value obtained by the blood pressure measurement method of the embodiment of the present invention, and the reason is that the difference between the blood pressure measurement result value obtained by the conventional method and the standard blood pressure measurement result value is larger. The above examples can demonstrate the improvement of the blood pressure measurement method of the embodiment of the present invention compared with the conventional method.

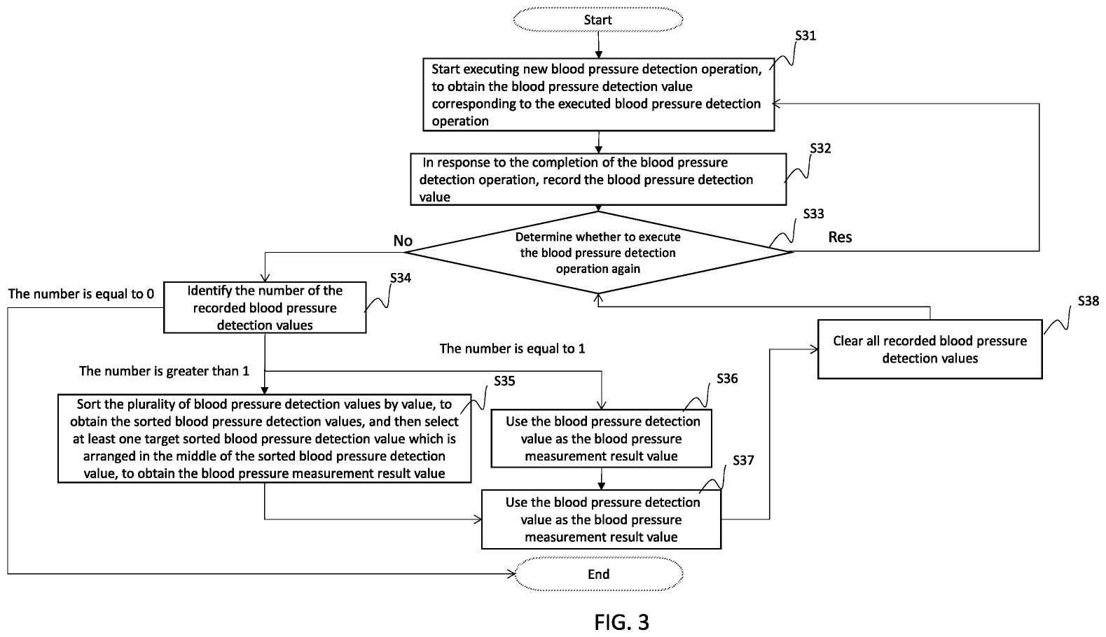

In another embodiment, the blood pressure measurement device 10 can be not provided with the input unit 130. In another embodiment, the control unit 110 can use another blood pressure measurement method to obtain the blood pressure measurement result value without using the first parameter. The details will be described below with reference to FIG. 3.

Please refer to FIG. 3, which is a flowchart of a blood pressure measurement method of another embodiment of the present invention. The blood pressure measurement method of another embodiment includes step S31 to S38.

As shown in FIG. 3, in a step S31, the control unit 110 instructs the detector 120 to start executing new blood pressure detection operation, so as to obtain the blood pressure detection value corresponding to the executed blood pressure detection operation. For example, in a predetermined time after the blood pressure measurement device 10 is powered on or the power management unit 160 receives a power-on signal from a power switch of the blood pressure measurement device 10, the control unit 110 instructs the detector 120 to start executing new blood pressure detection operation. For example, the detector 120 can comprise a sensor such as a magnetic sensor or a light sensor, to determine whether the user has an intent to execute the blood pressure detection operation. When the detector 120 determines that the user has the intent to execute the blood pressure detection operation, the detector 120 starts executing new blood pressure detection operation. As above, every time the blood pressure detection operation is completed, the detector 120 outputs the blood pressure detection value corresponding to the blood pressure detection operation completed this time, to the control unit 110.

Next, in a step S32, in response to the completion of the blood pressure detection operation, the control unit 110 records the blood pressure detection value. As above, in response to the completion of this blood pressure detection operation, the control unit 110 temporarily stores the blood pressure detection value to the storage unit 150 upon receipt of the blood pressure detection value.

In a step S33, the control unit 110 determines whether to execute the blood pressure detection operation again. More specifically, in the beginning of the step S33, the control unit 110 outputs a message, through the output unit 140, to inform the user that the previous blood pressure detection operation is completed, and the control unit 110 starts counting a trigger time until a trigger signal is received. The user can transmit the trigger signal by short pressing the power switch of the blood pressure measurement device 10, to input the intention thereof to execute the new blood pressure detection operation again. For example, the detector 120 can determine, through the sensor, that user's arm does not leave the detector 120, and after a short time, the detector 120 can automatically transmit the trigger signal to the control unit 110.

In response to the received trigger signal, the control unit 110 stops counting the trigger time. Next, the control unit 110 determines whether the trigger time is longer than an interval threshold value. In an embodiment, the interval threshold value can be 1 minute or 5 minutes, and the manufacturer can set the interval threshold value.

In response to the trigger time not longer than the interval threshold value, the control unit 110 determines to execute the blood pressure detection operation again, and the blood pressure measurement method returns to the step S31, the new blood pressure detection operation is executed again to obtain other blood pressure detection value.

In contrast, in response to the trigger time longer than the interval threshold value, the control unit 110 determines that the blood pressure detection operation is no longer executed. Next, in a step S34, the control unit 110 identifies the number of the recorded blood pressure detection values. More specifically, in a step S34, the control unit 110 identifies the number of the all blood pressure detection values temporarily stored in the storage unit 150, and the control unit 110 can execute different steps to obtain the blood pressure measurement result value according to the different number, in subsequent operation.

In response to the number higher than 1, the blood pressure measurement method continues to step S35, and the control unit 110 sorts the plurality of blood pressure detection values by value, to obtain the sorted blood pressure detection values, and then selects at least one target sorted blood pressure detection value which is arranged in the middle of the sorted blood pressure detection value, to obtain the blood pressure measurement result value.

The step S35 is similar to the aforementioned step S23, the control unit 110 can determine the median of the N sorted blood pressure detection values by the median method, and takes the determined median as the blood pressure measurement result value, so the detailed descriptions of the same parts are not repeated. However, the difference between the step S35 and the step S23 is that the number of the blood pressure detection values in the step S35 is two, that is, N can be 2.

Next, in a step S37, the control unit 110 instructs the output unit 140 to output the blood pressure measurement result value. The details of the step S37 are the same as the step S24 described above, so the detailed descriptions are omitted herein.

In response to the number equal to 1, the blood pressure measurement method continues to a step S36, the control unit 110 uses the blood pressure detection value as the blood pressure measurement result value. In other words, when the number of the temporarily stored blood pressure detection value is only one, the control unit 110 directly takes the temporarily stored blood pressure detection value as the blood pressure measurement result value, and the blood pressure measurement method continues to a step S37.

In another embodiment, after the blood pressure measurement result value is outputted, the blood pressure measurement method continues to a step S38. In the step S38, the control unit 110 clears all recorded blood pressure detection values. In other words, after a blood pressure measurement result value is outputted, all temporarily stored blood pressure detection values are deleted regardless of quantity of the temporarily stored blood pressure detection values. After the recorded blood pressure detection values are cleared, the blood pressure measurement method returns to the step S33. In response to the number being equal to 0, the control unit 110 is not enable to output any blood pressure measurement result value, and the control unit 110 determines the blood pressure measurement device 10 to be idle, and ends the overall flow of the blood pressure measurement method. At this time, the control unit 110 can instruct the power management unit 160 to turn off the blood pressure measurement device 10; alternatively, the control unit 110 waits for the next condition, for triggering the step S31, to be satisfied, and then re-executes the overall flow of the blood pressure measurement method from the step S31.

According to above-mentioned contents, the blood pressure measurement device and blood pressure measurement method of the embodiments of the present invention can perform multiple blood pressure detection operations, to obtain the multiple blood pressure detection values, and sort multiple plurality of blood pressure detection values to obtain the multiple sorted blood pressure detection values, and select at least one target sorted blood pressure detection value which is arranged in the middle of the sorted blood pressure detection values, so as to obtain the blood pressure measurement result value, thereby preventing the deviation problem resulted from the extreme values of the blood pressure measurement results obtained by the conventional averaging blood pressure detection operation.

The present invention disclosed herein has been described by means of specific embodiments. However, numerous modifications, variations and enhancements can be made thereto by those skilled in the art without departing from the spirit and scope of the disclosure set forth in the claims.

What is claimed is:

1. A blood pressure measurement device, comprising:
a control unit;
a detector coupled to the control unit and configured to measure a blood pressure of a user; and
an output unit coupled to the control unit and configured to output information;
wherein the control unit is configured to identify a first parameter, and the first parameter is configured to indicate the number of times for which a blood pressure detection operation should be executed;
wherein when the first parameter is higher than 2, the control unit instructs the detector to sequentially execute the blood pressure detection operation for N times according to the first parameter, so as to obtain the N blood pressure detection values, and a value of N is equal to the first parameter,
and the control unit sorts the N blood pressure detection values by value to obtain N sorted blood pressure detection values, and selects at least one target sorted blood pressure detection value which is arranged in the middle of the N sorted blood pressure detection values, so as to obtain a blood pressure measurement result value,
and the control unit instructs the output unit to output the blood pressure measurement result value.

2. The blood pressure measurement device according to claim 1, further comprising an input unit coupled to the control unit and configured to receive the first parameter, through an input operation;
wherein when the first parameter is equal to 1, the control unit instructs the detector to execute the blood pressure detection operation only once to obtain the blood pressure detection value, and uses the blood pressure detection value as the blood pressure measurement result value.

3. The blood pressure measurement device according to claim 1, wherein the operation that the control unit instructs the detector to sequentially execute the blood pressure detection operation for N times according to the first parameter, to obtain the N blood pressure detection values, comprises steps:
(a) the detector starts executing one blood pressure detection operation, to obtain the blood pressure detection value corresponding to the blood pressure detection operation executed currently;
(b) the control unit stores the obtained blood pressure detection value, to the storage unit, and accumulates a total number of executions; and
(c) the control unit determines whether the total number of executions is equal to N,
wherein when the total number of executions is not equal to N, the control unit instructs the detector to execute the blood pressure detection operation again, and the operation returns to the step (a);
wherein when the total number of executions is equal to N, the operation that the control unit instructs the detector to sequentially execute the blood pressure detection operation for N times according to the first parameter, to obtain the corresponding N blood pressure detection values, is ended.

4. The blood pressure measurement device according to claim 3, wherein the operation that the control unit selects the at least one target sorted blood pressure detection value which is arranged in the middle of the N sorted blood pressure detection values to obtain the blood pressure measurement result value, comprises steps:
when the first parameter is an even value, the control unit selects two target sorted blood pressure detection values which are arranged in the middle of the N sorted blood pressure detection values, and uses an average of the two target sorted blood pressure detection values as the blood pressure measurement result value;
when the first parameter is an odd value, the control unit selects the target sorted blood pressure detection value which is arranged in the middle of the N sorted blood pressure detection values, and uses the target sorted blood pressure detection value as the blood pressure measurement result value.

5. The blood pressure measurement device according to claim 3, wherein the operation that the control unit selects the at least one target sorted blood pressure detection value which is arranged in the middle of the N sorted blood pressure detection values to obtain the blood pressure measurement result value, comprises steps:
when the first parameter is an even value, the control unit selects two target sorted blood pressure detection values which are arranged in the middle of the N sorted blood pressure detection values, and uses an average of the two target sorted blood pressure detection values as the blood pressure measurement result value;
when the first parameter is an odd value lower than 5, the control unit selects a target sorted blood pressure detection value which is arranged in the middle of the N sorted blood pressure detection values, and uses the target sorted blood pressure detection value as the blood pressure measurement result value; and
when the first parameter is an odd value not lower than 5, the control unit selects three target sorted blood pressure detection values which are arranged in the middle of the N sorted blood pressure detection values, and uses an average of the three target sorted blood pressure detection values as the blood pressure measurement result value.

6. A blood pressure measurement method, comprising:
identifying a first parameter, wherein the first parameter indicates the number of times for which a blood pressure detection operation should be executed;
when the first parameter is higher than 2, using a detector to sequentially execute the blood pressure detection operation for N times according to the first parameter to obtain N blood pressure detection values, wherein a value of N is equal to the first parameter;
using a control unit to sort the N blood pressure detection values by value to obtain N sorted blood pressure detection values, and selecting at least one target sorted blood pressure detection value which is arranged in the middle of the N sorted blood pressure detection values, so as to obtain a blood pressure measurement result value; and
using an output unit to output the blood pressure measurement result value.

7. The blood pressure measurement method according to claim 6, wherein the first parameter is inputted through an input operation, and the blood pressure measurement method further comprises:
when the first parameter is equal to 1, using the detector to execute the blood pressure detection operation only once to obtain the blood pressure detection value, and using the blood pressure detection value as the blood pressure measurement result value for output.

8. The blood pressure measurement method according to claim 6, wherein the step of using the detector to sequentially execute the blood pressure detection operations for N times according to the first parameter to obtain the corresponding N blood pressure detection values, further comprises:
(a) using the detector to start executing the blood pressure detection operation to obtain the blood pressure detection value corresponding to the blood pressure detection operation executed currently;
(b) using the control unit to store the obtained blood pressure detection value to the storage unit, and accumulate a total number of executions; and
(c) using the control unit to determine whether the total number of executions is equal to N;
wherein when the total number of executions is not equal to N, using the control unit to instruct the detector to execute the blood pressure detection operation again, and returning to the step (a);
wherein when the total number of executions is equal to N, ending the step of using the detector to sequentially execute the blood pressure detection operation for N times according to the first parameter to obtain the N blood pressure detection values.

9. The blood pressure measurement method according to claim 8, wherein the step of using the control unit to select at least one target sorted blood pressure detection value which is arranged in the middle of the N sorted blood pressure detection values to obtain the blood pressure measurement result value, further comprises:
when the first parameter is an even value, using the control unit to select two target sorted blood pressure detection values which are arranged in the middle of the N sorted blood pressure detection values, and use an average of the two target sorted blood pressure detection values as the blood pressure measurement result value; and
when the first parameter is an odd value, using the control unit to select the target sorted blood pressure detection value which is arranged in the middle of the N sorted blood pressure detection values, and use the target sorted blood pressure detection value as the blood pressure measurement result value.

10. The blood pressure measurement method according to claim 8, wherein the step of selecting the at least one target sorted blood pressure detection value which is arranged in the middle of the N sorted blood pressure detection values to obtain the blood pressure measurement result value, further comprises:
when the first parameter is an even value, using the control unit to select two target sorted blood pressure detection values which are arranged in the middle of the N sorted blood pressure detection values, and use an average of the two target sorted blood pressure detection values as the blood pressure measurement result value;
when the first parameter is an odd value lower than 5, using the control unit to select a target sorted blood pressure detection value which is arranged in the middle of the N sorted blood pressure detection values, and use the target sorted blood pressure detection value as the blood pressure measurement result value; and
when the first parameter is an odd value not lower than 5, using the control unit to select three target sorted blood pressure detection values which are arranged in the middle of the N sorted blood pressure detection values, and use an average of the three target sorted blood pressure detection values as the blood pressure measurement result value.

11. A blood pressure measurement device, comprising:
a control unit;
a detector coupled to the control unit and configured to measure a blood pressure of a user; and
an output unit coupled to the control unit and configured to output information;
wherein the control unit instructs the detector to start executing a blood pressure detection operation, to obtain a blood pressure detection value corresponding to the blood pressure detection operation, and in response to the completion of the blood pressure detection operation, the control unit records the blood pressure detection value;
wherein the control unit determines whether to execute the blood pressure detection operation again;
wherein in response to the determination that the blood pressure detection operation is no longer executed, the control unit identifies the number of the recorded blood pressure detection values;
wherein in response to the number higher than 1, the control unit sorts the blood pressure detection values by value to obtain the sorted blood pressure detection values, and selects at least one target sorted blood pressure detection value which is arranged in the middle of the sorted blood pressure detection value, to obtain the blood pressure measurement result value;
wherein the control unit instructs the output unit to output the blood pressure measurement result value.

12. The blood pressure measurement device according to claim 11, wherein the operation that the control unit selects at least one target sorted blood pressure detection value which is arranged in the middle of the sorted blood pressure detection value, to obtain the blood pressure measurement result value, further comprises:
when the number is an even value, the control unit selects two target sorted blood pressure detection values which are arranged in the middle of the sorted blood pressure detection value, and uses an average of the target sorted blood pressure detection values as the blood pressure measurement result value;
wherein when the number is an odd value, the control unit selects the target sorted blood pressure detection value which is arranged in the middle of the sorted blood pressure detection value, and uses the target sorted blood pressure detection value as the blood pressure measurement result value.

13. The blood pressure measurement device according to claim 11, wherein the operation that the control unit selects at least one target sorted blood pressure detection value which is arranged in the middle of the sorted blood pressure detection value, to obtain the blood pressure measurement result value, further comprises:
when the number is an even value, the control unit selects two target sorted blood pressure detection values which are arranged in the middle of the sorted blood pressure detection value, and uses an average of the two target sorted blood pressure detection values as the blood pressure measurement result value;
wherein when the number is an odd value lower than 5, the control unit selects the target sorted blood pressure detection value which is arranged in the middle of the sorted blood pressure detection value, and uses the target sorted blood pressure detection value as the blood pressure measurement result value;
wherein when the first parameter is an odd value not lower than 5, the control unit selects three target sorted blood pressure detection values which are arranged in the middle of the sorted blood pressure detection value, and uses an average of the three target sorted blood pressure detection values as the blood pressure measurement result value.

14. The blood pressure measurement device according to claim 11, wherein in response to the number equal to 1, the control unit uses the blood pressure detection value as the blood pressure measurement result value.

15. The blood pressure measurement device according to claim 11, wherein after outputting the blood pressure measurement result value, the control unit clears the recorded blood pressure detection values, and re-executes the operation of determining whether to execute the blood pressure detection operation again.

16. The blood pressure measurement device according to claim 11, wherein the operation that the control unit determines whether to execute the blood pressure detection operation again, further comprises:
the control unit starts to count a trigger time until the control unit receives a trigger signal;
the control unit determines whether the trigger time is higher than an interval threshold value;
in response to the trigger time higher than the interval threshold value, the control unit determines not to execute the blood pressure detection operation again; and
in response to the trigger time not higher than the interval threshold value, the control unit determines to execute the blood pressure detection operation again.
